(19) United States
(12) Patent Application Publication
(10) Pub. No.: US 2007/0134297 A1
(43) Pub. Date: Jun. 14, 2007

(54) DEVICE TO AID IN SEXUAL PLEASURE

(76) Inventor: Naqam Washington, (US)

Correspondence Address: HOWARD M COHN;PATENT ATTORNEY LLC, 21625 CHAGRIN BLVD #220, CLEVELAND, OH, 44122, US

(21) Appl. No.: 11/608,315

(22) Filed: Dec. 8, 2006

Related U.S. Application Data
(60) Provisional application No. 60/597,541, filed on Dec. 8, 2005.

(51) Int. Cl. A61K 47/00

(52) U.S. Cl. 424/439

(57) ABSTRACT

There is provided a dissolving strip adapted to be placed on a person's tongue to aid in giving oral sexual pleasure to both men and women. The strip comprises: a dissolvable, orally consumable, composition; and the strip is flexible and adapted to adhere to a surface in the mouth, usually the tongue. The strip dissolves in about 5 to 20 minutes and preferably in about 10 to 15 minutes.

### CROSS-REFERENCE TO RELATED APPLICATIONS

This application claims the benefit of U.S. Provisional Patent Application No. 60/597,541, filed Dec. 8, 2005 which is incorporated herein by reference.

### BACKGROUND

This invention relates generally to an easy to use, inexpensive and safe device to aid in giving oral sexual pleasure to both men and women.

In the past there have been a number of devices to aid in the oral pleasure for both men and women. However, none of the prior art devices were small, consumable, and safe.

For example, U.S. Pat. No. 6,244,269 discloses a two piece pliable, edible, flavored mouthpiece to bc worn when performing oral sexual activities. While this device is edible, it's not very small and functions differently from the present invention.

U.S. Pat. No. 5,657,765 discloses an erotic stimulator in the form of a tongue. While this stimulator can be placed on a tongue and used for oral pleasure, it is not small and consumable.

U.S. Pat. Nos. 6,419,903 and 6,923,981 disclose a rapidly dissolvable, orally consumable film composition for delivering breath freshening agents to the oral cavity. While these devices are edible and can be placed on the user's tongue, they are not designed to give oral sexual pleasure as with the present invention.

### ASPECTS AND SUMMARY OF THE INVENTION

It is an aspect of the present invention to provide a device to aid in giving oral sexual pleasure that overcomes the problems with the aforementioned prior art.

It is another object of the present invention to provide a device to aid in giving oral sexual pleasure made at least partially of an edible substance.

It is still another object of the present invention to provide a device to aid in giving oral sexual pleasure made at least partially of an edible substance in which there is no clean-up.

It is yet another object of the present invention to provide a device to aid in giving oral sexual pleasure made at least partially of an edible substance in which there is no waste.

It is a further object of the present invention to provide a device to aid in giving oral sexual pleasure made at least partially of an edible substance that is easy and economical to use and make.

It is a further object of the present invention to provide a device to aid in giving oral sexual pleasure made at least partially of a strip of edible substance that has a plurality of ridges and/or bumps projecting above the upper surface of the strip in any shape or configuration.

According to the invention, there is provided a dissolving strip adapted to be placed on a person's tongue to aid in giving oral sexual pleasure to both men and women. The strip comprises: a dissolvable, orally consumable, composition; and the strip is flexible and adapted to adhere to a surface in the mouth, usually the tongue. The strip dissolves in about 5 to 20 minutes and preferably in about 10 to 15 minutes.

Further according to the invention, the dissolving strip has a plurality of ridges projecting above the upper surface of the strip in any shape or configuration. The plurality of ridges projecting upward from the upper surface are preferably between about 1 to about 5 millimeters in height so that they melt away or dissolves in about 5 to 20 minutes.

Still further according to the invention, the dissolving strip has a plurality of protrusions projecting upward from the upper surface of the strip. The protrusions have a shape from the group comprising convex, mammilated, round and hemispherical shapes. The plurality of protrusions projecting upward from the upper surface are preferably between about 1 to about 5 millimeters so that they melt away or dissolves in about 5 to 20 minutes.

Yet further according to the invention, the dissolving strip has a length of about 40 to about 100 millimeters, a width of about 12 to 30 millimeters and a thickness of about 12 to 30 millimeters.

Still further according to the invention, the dissolving strip has a shape selected from the group consisting of triangular, circular, semicircular, hexagonal, octagonal, and quadrilateral.

According to the invention, there is provided a dissolving strip having a multi-layer construction. The multi-layer construction is formed of two layers, each of a different composition wherein one layer has a first flavor and the second layer has a different flavor. Alternatively, one layer has a first flavor and the second layer has a gummi composition. In another embodiment, the upper layer has a first flavor and the bottom layer includes has a breath freshener agent.

Also according to the invention, the multi-layer construction is formed of three layers, each of a different composition. The strip with a multi-layer construction dissolves in about 5 to 20 minutes.

Further according to the invention, the multi-layer construction strip has a plurality of ridges projecting above the upper surface of the strip in any shape or configuration. In addition or alternatively thereto the dissolving strip has a plurality of protrusions projecting upward from the upper surface of the strip. The protrusions have a shape from the group comprising convex, mammilated, round and hemispherical shapes.

Other objects, features and advantages of the invention will become apparent in light of the following description thereof.

### BRIEF DESCRIPTIONS OF THE DRAWINGS

The structure, operation, and advantages of the present invention will become further apparent upon consideration of the following description taken in conjunction with the accompanying figures (FIGS.). The figures are intended to be illustrative, not limiting.

Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, often both reference numerals and legends (labels, text descriptions) may be used to identify elements. If legends are provided, they are intended merely as an aid to the reader, and should not in any way be interpreted as limiting.

Often, similar elements may be referred to by similar numbers in various figures (FIGS.) of the drawing, in which case typically the last two significant digits may be the same, the most significant digit being the number of the drawing figure (FIG.).

### BRIEF DESCRIPTION OF THE DRAWINGS

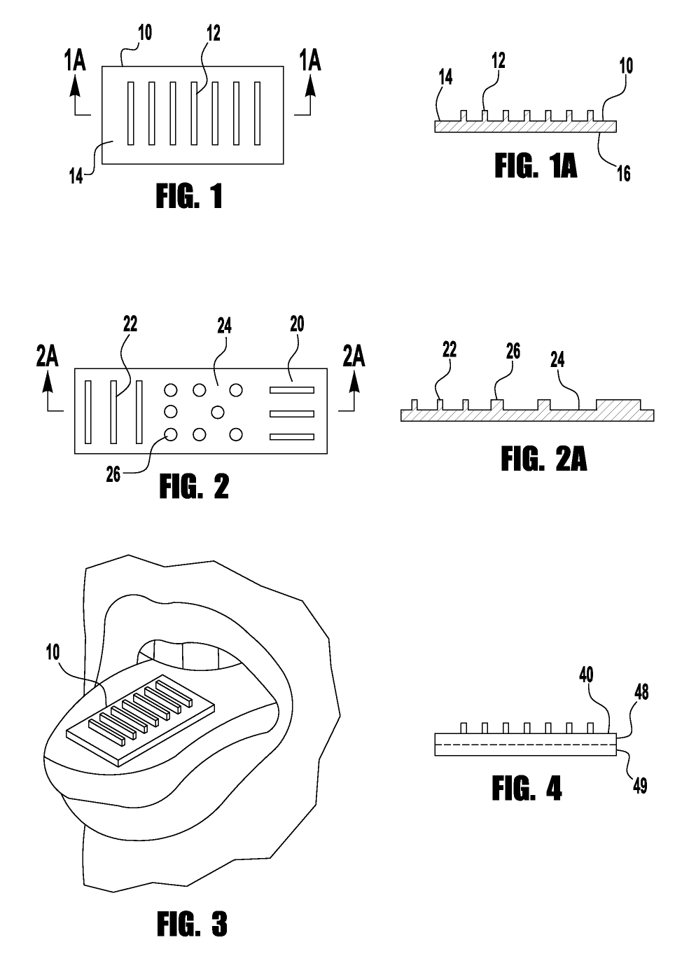

FIG. 1 is a plan view of a first embodiment of a device to aid in oral pleasure incorporating ridges, in accordance with the present invention;

FIG. 1A is cross-sectional view through line 1A-1A of FIG. 1;

FIG. 2 is a plan view of a second embodiment of a device to aid in oral pleasure incorporating ridges and bumps, in accordance with the present invention;

FIG. 2A is cross-sectional view through line 2A-2A of FIG. 2;

FIG. 3 is a plan view of the first embodiment of a device to aid in oral pleasure incorporating ridges placed on the upper surface of a tongue, in accordance with the present invention; and

FIG. 4 is a cross-sectional side view of a third embodiment of a device to aid in oral pleasure incorporating ridges, in accordance with the present invention.

### DETAILED DESCRIPTION OF THE PREFERRED EMBODIMENTS

The present invention is a novel solution to aid in orally providing sexual pleasure for both men and women. It provides an easy to use, inexpensive and safe device to provide a man or woman with erotic pleasure.

Referring to FIG. 1, there is illustrated a first embodiment of the present invention comprising a dissolving strip 10 that is placed on a person's tongue to provide a woman with oral pleasure. The strip 10 is formed of a relatively, rapidly dissolvable, orally consumable film composition. Moreover, the strip is flexible.

While the strip 10 can be of any composition, an exemplary strip can comprise hydroxyalkylmethylcellulose as a strip forming agent combined with starch and a flavoring agent. The strip can also comprise water, additional strip forming agents, plasticizing agents, flavoring agents, antimalodor agents, surfactants, emulsifying agents, coloring agents, sweeteners and fragrances. Besides the strips of the present invention having any desired flavor, they also have any desired color.

Flavor agents that can be used to prepare the taste and freshening strip of the present invention include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. These flavor agents can be used individually or in admixture. Commonly used flavors include mints such as peppermint, artificial vanilla, cinnamon derivatives, and various fruit flavors, whether employed individually or in admixture. Generally, any flavoring or food additive, such as those described in Chemicals Used in Food Processing, publication 1274 by the National Academy of Sciences, pages 63-258, may be used. The amount of flavoring agent employed is normally a matter of preference subject to such factors as flavor type, individual flavor, and strength desired. Generally the flavoring is incorporated in the strip of the present invention in an amount ranging from about 2 to about 10% by weight and preferably about 5 to about 8% by weight.

Sweeteners useful in the practice of the present invention include both natural and artificial sweeteners. Suitable sweetener include water soluble sweetening agents such as monosaccharides, disaccharides and polysaccharides such as xylose, ribose, glucose (dextrose), mannose, glatose, fructose (levulose), sucrose (sugar), maltose, water soluble artificial sweeteners such as the soluble saccharin salts, i.e., sodium or calcium saccharin salts, cyclamate salts dipeptide based sweeteners, such a L-aspartic acid derived sweeteners, such as L-aspartyl-L-phenylalaine methyl ester (aspartame). The compositions of the present invention can also contain coloring agents or colorants. The coloring agents are used in amounts effective to produce the desired color and include natural food colors and dyes suitable for food, drug and cosmetic applications.

These colorants are known as FD&C dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 3 comprises a 15 triphenylmethane dye and is the monosodium salt of 4-[4-N-ethyl-p-sulfobenzylamino) diphenyl-methylene]-[1-N-ethy 1-N-sulfonium benzyl)-2,5-cyclo-hexadienimine ]. A full recitation of all FD&C and D&C dyes and their corresponding chemical structures may be found in the Kirk-Othrner Encyclopedia of Chemical Technology, Volume 5, Pages 857-884, which text is accordingly incorporated herein by reference.

It is also within the terms of the present invention to make the entire strip 10 from a soft, deformable, dissolvable and edible material. For example, a suitable material that can be used is a gummi candy commonly made, for example, from corn syrup, sugar, gelatine and citric acid.

During oral sex, the gummi candy softens with the saliva of a person. Because of the sticky nature of strip 10 and because of the friction forces, strip 10 can remain on a user's tongue during any sliding action against a vagina or a penis. After oral sex, the gummi candy can be chewed and swallowed.

It is also within the terms of the present invention to incorporate active breath freshening agents into any of the strip compositions of any of the disclosed embodiments of the present invention to form the breath freshening strips. The active ingredients include zinc gluconate, zinc citrate and/or alpha ionone. These agents function in masking mouth odor and reducing volatile odor causing bacterial sulfur compounds. These agents may be incorporated in the strip composition of the present invention at a concentration of about 0.1 to about 2.0% by weight and preferably about 0.15 to about 0.5% by weight.

The strip 10 is shaped and sized to be placed in the oral cavity. The strip 10 is flexible and adheres by the bottom surface 16 to a surface in the mouth, usually the tongue, as shown in FIG. 3, and dissolves in about 5 to 20 minutes and preferably about 10 to 15 minutes. It is also within the terms of the invention to adhere the strip to the roof of the mouth.

The width and length of the strip 10 corresponds to about the size of one postage stamp.

That is generally about 12 to 30 millimeters in width and about 20 to about 50 millimeters in length. The strip 10 has a thickness of about 1 to about 5 millimeters and preferably about 2 to about 4 millimeters so that it melts or dissolves in about 5 to 20 minutes and preferably about 10 to 15 minutes. While the strip can be rectangular in shape, it can have any shape such as but not limited to triangular, circular, semicircular, hexagonal, octagonal, quadrilateral, or any other shape. Also, the size of the strip is not limited except that it can fit into the mouth of the user.

As shown in FIGS. 1 and 1A, the strip 10 can include a plurality of elongated salient ridges 12 projecting above the upper surface 14 of the strip. While the ridges 12 are shown extending parallel to one another, it is in the terms for the invention to form the ridges in any shape or configuration. The height of the ridges 12 are preferably between about 1 to about 5 millimeters so that they melt away or dissolves in about 5 to 20 minutes and preferably about 10 to 15 minutes.

Referring to FIG. 2, there is illustrated a second embodiment of the present invention comprising a dissolving strip 20 that is placed on a person's tongue to provide a man or woman with erotic pleasure. The strip 20 is substantially identical to strip 10 of FIG. 1 except for the size. The strip 20 is shaped and sized to be placed on the tongue and dissolves in about 5 to 20 minutes and preferably about 10 to 15 minutes.

The width and length of the strip 20 corresponds to about the size of two postage stamps disposed end to end. That is, about 12 to 30 millimeters in width and about 40 to 100 millimeters in length. The strip 20 has a thickness of about 1 to about 5 millimeters and preferably about 2 to about 4 millimeters so that it melts or dissolves in about 5 to 20 minutes and preferably about 10 to 15 minutes. As with the strip 10 of the first embodiment, the strip 20 can be rectangular in shape, and be of any shape such as but not limited to triangular, circular, semicircular, hexagonal, octagonal, quadrilateral, or any other shape. Also, the size of the strip 20 is not limited except that it can fit into the mouth of the user.

As shown in FIGS. 2 and 2A, a strip 20, which is essentially formed of the same materials as strip 10, can include a plurality of ridges 22 projecting above the upper surface 24 of the strip 20. Typically the lower surface 28 is flat and adapted to rest on a user's tongue, as generally shown in FIG. 3. While the ridges 12 are shown extending parallel to one another, it is in the terms for the invention to form the ridges in any shape or configuration, such as perpendicular to each other as shown. Also, a plurality of salient bumps 26 in any size and configuration can be provided on the upper surface 24 of strip 20 with or without the ridges 22. For example, the protrusions 26 can have a convex, mammilated, round or hemispherical shape to name a few. The protrusions are not limited to these shapes and it is within the terms of the invention to use a protrusion 26 of any desired shape.

The height of the ridges 12 and protrusions 26 are preferably between about 1 to about 5 millimeters so that they melt away or dissolve in about 5 to 20 minutes and preferably about 10 to 15 minutes.

As shown in FIG. 2, the strip 10 can include a plurality of ridges 22 projecting above the upper surface 24 of the strip 20. While the ridges 12 are shown extending parallel to one another, it is in the terms for the invention to form the ridges in any shape or configuration. Also, a plurality of bumps 26 in any size and configuration can be provided on the strip with or without the ridges 22.

FIG. 3 illustrates a strip 30, which can be any of the strips of the present invention disposed on a tongue.

Referring to FIG. 4, there is shown another embodiment of the present invention wherein a strip 40 has a multi-layer construction. For example, the strip 40 can be formed with two layers 48 and 49. Strip 40 can be constructed with a shape and dimensions corresponding to strip 10 and strip 20. The upper layer 48 can be of one flavor, such as an oil and the lower layer 49 can be of a different flavor than the upper layer and include a sweetener or a breath freshener agent of the type described herein after. It is also within the terms of the invention to make one of the layers, probably the lower layer 49 of gummi composition and the upper layer 48 of a different composition. Moreover, it is within the terms of the present invention to use three or more layers as desired.

It is also within the terms of the present invention to make the layers of different textures, such as rough or smooth.

Although the invention has been illustrated and described in detail in the drawings and foregoing description, the same is to be considered as illustrative and not restrictive in character—it being understood that only preferred embodiments have been shown and described, and that all changes and modifications that come within the spirit of the invention are desired to be protected. Undoubtedly, many other “variations” on the “themes” set forth hereinabove will occur to one having ordinary skill in the art to which the present invention most nearly pertains, and such variations are intended to be within the scope of the invention, as disclosed herein.

1. A dissolving strip adapted to be placed on a person's tongue to aid in giving oral sexual pleasure to both men and women, the strip comprising:
a dissolvable, orally consumable, composition; and
the strip being flexible and adapted to adhere to a surface in the mouth, usually the tongue.

2. The dissolving strip of claim 1 wherein the strip dissolves in about 5 to 20 minutes.

3. The dissolving strip of claim 2 wherein the strip dissolves in about 10 to 15 minutes.

4. The dissolving strip of claim 2 wherein the strip has a plurality of ridges projecting upward from the upper surface of the strip in any shape or configuration.

5. The dissolving strip of claim 4 wherein the plurality of ridges projecting upward from the upper surface are preferably between about 1 to about 5 millimeters in height so that they melt away or dissolves in about 5 to 20 minutes.

6. The dissolving strip of claim 2 wherein the strip has a plurality of protrusions projecting upward from the upper surface of the strip, said protrusions having a shape from the group comprising convex, mammilated, round and hemispherical shapes.

7. The dissolving strip of claim 6 wherein the plurality of protrusions project upward from the upper surface are preferably between about 1 to about 5 millimeters in height so that they melt away or dissolves in about 5 to 20 minutes.

8. The dissolving strip of claim 1 wherein the strip has a length of about 40 to about 100 millimeters.

9. The dissolving strip of claim 8 wherein the strip has a width of about 12 to 30 millimeters.

10. The dissolving strip of claim 8 wherein the strip has a thickness of about 12 to 30 millimeters.

11. The dissolving strip of claim 1 wherein the strip has a shape selected from the group consisting of triangular, circular, semicircular, hexagonal, octagonal, and quadrilateral.

12. The dissolving strip of claim 1 wherein the strip has a multi-layer construction.

13. The dissolving strip of claim 12 wherein the multi-layer construction is formed of two layers, each of a different composition.

14. The dissolving strip of claim 13 wherein one layer has a first flavor and the second layer has a different flavor.

15. The dissolving strip of claim 13 wherein one layer has a first flavor and the second layer has a gummi composition.

16. The dissolving strip of claim 13 wherein the upper layer has a first flavor and the bottom layer includes has a breath freshener agent.

17. The dissolving strip of claim 13 wherein the multi-layer construction is formed of three layers, each of a different composition.

18. The dissolving strip of claim 13 wherein the strip dissolves in about 5 to 20 minutes.

19. The dissolving strip of claim 13 wherein the strip has a plurality of ridges projecting above the upper surface of the strip in any shape or configuration.

20. The dissolving strip of claim 13 wherein the strip has a plurality of protrusions projecting upward from the upper surface of the strip, said protrusions having a shape from the group comprising convex, mammilated, round and hemispherical shapes.
